(19) United States
(12) Patent Application Publication
(10) Pub. No.: US 2026/0306437 A1
(43) Pub. Date: Oct. 8, 2026

(54) MOISTURE-PROOF ACETYL L-CARNITINE

(71) Applicant: NANJING NUTRABUILDING BIO-TECH CO., LTD., Nanjing (CN)

(72) Inventors: Wei WANG, Nanjing (CN); Chengfei WANG, Nanjing (CN); Shawn WELLS, Frisco, TX (US); Kylin LIAO, Nanjing (CN)

(21) Appl. No.: 19/682,510

(22) Filed: May 20, 2026

Related U.S. Application Data
(63) Continuation-in-part of application No. PCT/CN2024/132547, filed on Nov. 18, 2024.

(30) Foreign Application Priority Data
Nov. 23, 2023 (WO) PCT/CN2023/133610

(51) Int. Cl. A61K 31/205; A61K 9/16

(52) U.S. Cl. CPC A61K 31/205; A61K 9/1617; A61K 9/1623; A61K 9/1652

(57) ABSTRACT

The invention provides a moisture-proof acetyl L-carnitine or hydrochloride thereof and a preparation method thereof. Through the auxiliary material selection and preparation method treatment of raw materials of acetyl L-carnitine or hydrochloride thereof, the problems of strong moisture absorption and deliquescence when exposed to air are solved, and excellent moisture-proof and deliquescence-resistant effects are achieved, the stability of acetyl L-carnitine or hydrochloride thereof is improved, and high applicability is brought to users. In addition, the preparation process is simple, the production cost is low and it is environmentally friendly.

### CROSS REFERENCE TO RELATED APPLICATION

This application is a continuation-in-part application of International Patent Application No. PCT/CN2024/132547, filed on Nov. 18, 2024, which claims the priority of the International Application No. PCT/CN2023/133610, filed on Nov. 23, 2023, the contents of all of which are incorporated herein by reference in their entirety.

### FIELD OF THE INVENTION

The invention belongs to the technical field of dietary supplements and/or preparations; and particularly relates to a moisture-proof acetyl L-carnitine or hydrochloride thereof and a preparation method thereof.

### BACKGROUND OF THE INVENTION

Acetyl L-Carnitine is an esterified product of L-carnitine, which is white powder, soluble in water and insoluble in acetone. Acetyl L-carnitine is a natural substance existing in the body, especially in muscle, brain and sperm. It participates in a series of important metabolic processes in the human body and has good activity on the brain and reproductive system. The main physiological functions of acetyl L-carnitine or hydrochloride thereof include reducing blood lipid, improving human exercise ability and losing weight; protecting cells; preventing lactic acid accumulation; promoting the energy utilization of substances such as carbohydrates; improving the number and vitality of sperm.

However, acetyl L-carnitine or hydrochloride thereof is very easy to absorb moisture, so it often causes moisture absorption and brings a lot of inconvenience in transportation, storage and application. Therefore, it is urgent to develop a moisture-proof product of acetyl L-carnitine or hydrochloride thereof to solve the problems of easy moisture absorption, and this product will have a very broad market prospect.

### SUMMARY OF THE INVENTION

In order to solve the above problems, the present invention provides a moisture-proof acetyl L-carnitine or hydrochloride thereof and a preparation method thereof. Through the auxiliary material selection and preparation method treatment of raw materials of acetyl L-carnitine or hydrochloride thereof, the problems of strong moisture absorption and deliquescence when exposed to air are solved, and excellent moisture-proof and deliquescence-resistant effects are achieved, the stability of acetyl L-carnitine or hydrochloride thereof is improved, and at the same time, the requirement of high content is met, and great convenience is brought to transportation, storage and application scenes, and high applicability is brought to users. In addition, the preparation process of the invention is simple and the production cost is low. Besides, no organic solvent is involved in the production process, which is environmentally friendly, and the product has no risk of solvent residue.

In one aspect, the invention provides a moisture-proof acetyl L-carnitine or hydrochloride thereof, wherein the moisture-proof acetyl L-carnitine or hydrochloride thereof comprises, by weight, not less than 10% of acetyl L-carnitine, hydrochloride thereof, or the mixture of both.

In some embodiments, the moisture-proof acetyl L-carnitine or hydrochloride thereof comprises, by weight, not less than 15%, 20%, 25%, 30%, 35%, 40%, 45%, 50% of acetyl L-carnitine, hydrochloride thereof, or the mixture of both. In some embodiments, the moisture-proof acetyl L-carnitine or hydrochloride thereof comprises, by weight, not less than 12%, 14%, 16%, 18%, 22%, 24%, 26%, 28%, 32%, 34%, 36%, 38%, 42%, 44%, 46%, 48%, 52%, 55%, 58%, 60%, 62%, 65%, 68%, 70%, 72%, 75%, 78%, 80%, 85%, 90%, 95%, 97%, 99% of acetyl L-carnitine, hydrochloride thereof, or the mixture of both.

In some embodiments, the moisture-proof acetyl L-carnitine or hydrochloride thereof comprises, by weight, 10-99%, 15-98%, 20-97%, 25-96%, 30-95%, 35-94%, 40-93%, 45-92%, 50-90% of acetyl L-carnitine, hydrochloride thereof, or the mixture of both. In some embodiments, the moisture-proof acetyl L-carnitine or hydrochloride thereof comprises, by weight, 10-99%, 11-98%, 12-97%, 15-96%. 18-95%, 20-95%, 25-92%, 35-90% of acetyl L-carnitine, hydrochloride thereof, or the mixture of both.

In some embodiments, the moisture-proof acetyl L-carnitine or hydrochloride thereof comprises, by weight, 20-98% of acetyl L-carnitine, hydrochloride thereof, or the mixture of both; 1-60% of one or more auxiliary materials selected from microcrystalline cellulose, compressible starch, lactose or mannitol; 1-20% of one or more auxiliary materials selected from silicon dioxide, magnesium stearate, calcium hydrogen phosphate, calcium silicate, micro-powder silica gel.

In some embodiments, the moisture-proof acetyl L-carnitine or hydrochloride thereof comprises, by weight, 40-90% of acetyl L-carnitine, hydrochloride thereof, or the mixture of both; 2-50% of one or more auxiliary materials selected from microcrystalline cellulose, mannitol; 2-10% of one or more auxiliary materials selected from silicon dioxide, magnesium stearate.

In some embodiments, the moisture-proof acetyl L-carnitine or hydrochloride thereof comprises, by weight, 50-80% of acetyl L-carnitine, hydrochloride thereof, or the mixture of both; 5-45% of one or more auxiliary materials selected from microcrystalline cellulose, mannitol; 2-5% of magnesium stearate.

In some embodiments, the moisture-proof acetyl L-carnitine or hydrochloride thereof is in granular or powdery form.

In some embodiments, the particle size of the moisture-proof acetyl L-carnitine or hydrochloride thereof is 10-100 meshes. In some embodiments, the particle size of the moisture-proof acetyl L-carnitine or hydrochloride thereof is 20-80 meshes.

In some embodiments, the moisture-proof acetyl L-carnitine or hydrochloride thereof is formulated into nutritional supplement, food, beverage or animal feed.

In some embodiments, the moisture-proof acetyl L-carnitine or hydrochloride thereof is prepared into solid preparation or liquid preparation.

In some embodiments, the passing rate of the moisture-proof acetyl L-carnitine or hydrochloride thereof through a 24-mesh sieve is not less than 70%, 75%, 80%, 85% after standing for a period of time. In some embodiments, the passing rate of the moisture-proof acetyl L-carnitine or hydrochloride thereof through a 24-mesh sieve is not less than 80%, 85%, 87%, 89% after being placed (standing) for 0.5 h. In some embodiments, the passing rate of the moisture-proof acetyl L-carnitine or hydrochloride thereof through a 24-mesh sieve is not less than 75%, 80%, 81% after standing for 3 h. In some embodiments, the passing rate of the moisture-proof acetyl L-carnitine or hydrochloride thereof through a 24-mesh sieve is not less than 65%, 70%, 75% after standing for 5 h. In some embodiments, the passing rate of the moisture-proof acetyl L-carnitine or hydrochloride thereof through a 24-mesh sieve is not less than 50%, 60%, 65%, 70% after standing for 7 h.

In some embodiments, the moisture-proof acetyl L-carnitine or hydrochloride thereof is placed under high humidity conditions, e.g., at temperature of 40° C.±2° C. and relative humidity of 75%±5%.

In some embodiments, the increase of moisture content of the moisture-proof acetyl L-carnitine or hydrochloride thereof is not more than 1.0%, 2.0%, 4.0%, 5.0% after standing for a period of time. In some embodiments, the increase of moisture content of the moisture-proof acetyl L-carnitine or hydrochloride thereof is not more than 0.8%, 1.0%, 1.5% after standing for 0.5 h. In some embodiments, the increase of moisture content of the moisture-proof acetyl L-carnitine or hydrochloride thereof is not more than 2.0%, 2.5%, 3.0% after standing for 1 h. In some embodiments, the increase of moisture content of the moisture-proof acetyl L-carnitine or hydrochloride thereof is not more than 3.8%, 4.0%, 4.5% after standing for 2 h. In some embodiments, the increase of moisture content of the moisture-proof acetyl L-carnitine or hydrochloride thereof is not more than 4.8%, 5.0%, 5.5% after standing for 5 h.

In some embodiments, the moisture-proof acetyl L-carnitine or hydrochloride thereof is placed under high humidity conditions, e.g., at temperature of 40° C.±2° C. and relative humidity of 75%±5%.

In some embodiments, the repose angle of the moisture-proof acetyl L-carnitine or hydrochloride thereof is not more than 45°. In some embodiments, the repose angle of the moisture-proof acetyl L-carnitine or hydrochloride thereof is not more than 48°, 50°.

In another aspect, the invention provides a method for preparing moisture-proof acetyl L-carnitine or hydrochloride thereof, which comprises mixing the following components of prescribed amounts: acetyl L-carnitine, hydrochloride thereof, or the mixture of both; one or more auxiliary materials selected from microcrystalline cellulose, compressible starch, lactose or mannitol; one or more auxiliary materials selected from silicon dioxide, magnesium stearate, calcium hydrogen phosphate, calcium silicate, micro-powder silica gel; granulating; to obtain the moisture-proof acetyl L-carnitine or hydrochloride thereof.

In some embodiments, the moisture-proof acetyl L-carnitine or hydrochloride thereof comprises, by weight, 40-90% of acetyl L-carnitine, hydrochloride thereof, or the mixture of both; 2-50% of one or more auxiliary materials selected from microcrystalline cellulose, mannitol; 2-10% of one or more auxiliary materials selected from silicon dioxide, magnesium stearate.

In some embodiments, the moisture-proof acetyl L-carnitine or hydrochloride thereof comprises, by weight, 50-80% of acetyl L-carnitine, hydrochloride thereof, or the mixture of both; 5-45% of one or more auxiliary materials selected from microcrystalline cellulose, mannitol; 2-5% of magnesium stearate.

In some embodiments, the granulation is one of wet granulation, spray granulation, melt granulation and dry granulation.

In some embodiments, the granulation is dry granulation.

In some embodiments, the moisture-proof acetyl L-carnitine or hydrochloride thereof prepared by the method is used for preparing food, beverage, nutrition, skin care products or cosmetics.

In some embodiments, the method comprises a sieving step. In some embodiments, sieving acetyl L-carnitine, hydrochloride thereof, or the mixture of both; and/or mannitol with a 10-100 mesh sieve. In some embodiments, sieving acetyl L-carnitine, hydrochloride thereof, or the mixture of both; and/or mannitol with a 20-80 mesh sieve.

This summary is provided to introduce the selection of concepts in a simplified form, which are further described in the detailed description below. Summary is not intended to identify key features or essential features of the claimed subject matter, nor is it intended to be used to limit the scope of the claimed subject matter.

### DETAILED DESCRIPTION OF THE INVENTION

Reference will now be made in detail to the preferred embodiments of the present disclosure, examples of which are further illustrated. While the present disclosure will be described in conjunction with the preferred embodiments, it will be understood that they are not intended to limit the present disclosure to these embodiments. To the contrary, the present disclosure is intended to cover alternatives, modifications and equivalents, which may be included within the spirit and scope of the present disclosure as defined by the claims.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

### EXAMPLES

In the present disclosure, unless otherwise specified, scientific and technical terms used herein have the meanings commonly understood by those skilled in the art. Moreover, the laboratory operation steps used herein are all routine steps widely used in the corresponding fields.

The raw materials or reagents used in the present disclosure are commercially available unless otherwise specified. All reagents are commercial grade and used according to the received standards.

On the one hand, the present invention provides a moisture-proof acetyl L-carnitine, hydrochloride thereof, or the mixture of both. The moisture-proof acetyl L-carnitine, hydrochloride thereof, or the mixture of both of the invention has excellent moisture-proof and deliquescence resistance, improves the stability of acetyl L-carnitine or hydrochloride thereof, and brings high applicability to users. Acetyl L-carnitine or hydrochloride thereof used in the present invention can be prepared and obtained by known methods.

On the other hand, the invention screens raw materials of acetyl L-carnitine or hydrochloride thereof. In addition, wet granulation, spray granulation, melt granulation, dry granulation and other granulation methods were screened, and the dry granulation is preferred. The adopted dry granulation is suitable for the formula of the invention and will not cause bed collapse. The process energy consumption is low, the production cost is low; and the preparation process is free of solvent intervention, the production process is green and environment-friendly, and no waste gas or wastewater is generated. The addition of microcrystalline cellulose, mannitol and other auxiliary materials can achieve good anti-caking effect in this formula; auxiliary materials such as magnesium stearate have good anti-caking and anti-sticking effects, and the combination of these materials has the advantages of high production yield, remarkable moisture absorption resistance and economical and easily available auxiliary materials. In the method, a 10-100 mesh, preferably a 20-80 mesh sieve, can be used to granulate to obtain the moisture-proof acetyl L-carnitine or hydrochloride thereof.

Through the screening of suitable auxiliary materials and granulating methods, the invention realizes the preparation of high-content moisture-proof acetyl L-carnitine, hydrochloride thereof, or the mixture of both. The prepared moisture-proof acetyl-L-carnitine, hydrochloride thereof, or the mixture of both has excellent moisture-proof and deliquescence resistance, improves the stability of acetyl-L-carnitine or hydrochloride thereof, and brings high applicability to users.

The following examples are illustrative of selected embodiments of the present disclosure and are not meant to limit the scope of the present disclosure.

### Example 1 Preparation of Moisture-Proof Acetyl L-Carnitine

A moisture-proof acetyl L-carnitine comprises the following components in percentage by weight:

| | |
|---|---|
| acetyl L-carnitine | 50% |
| microcrystalline cellulose | 45% |
| magnesium stearate | 5%. |

Acetyl L-carnitine was sieved with a 30-mesh sieve, acetyl L-carnitine, microcrystalline cellulose and magnesium stearate of prescribed amounts were weighed and put into a three-dimensional mixer, dry granulation was carried out, to obtain the moisture-proof acetyl L-carnitine.

### Example 2 Preparation of Moisture-Proof Acetyl L-Carnitine

A moisture-proof acetyl L-carnitine comprises the following components in percentage by weight:

| | |
|---|---|
| acetyl L-carnitine | 60% |
| mannitol | 20% |
| microcrystalline cellulose | 15% |
| magnesium stearate | 5%. |

Acetyl L-carnitine and mannitol were sieved with a 30-mesh sieve, acetyl L-carnitine, mannitol, microcrystalline cellulose and magnesium stearate of prescribed amounts were weighed and put into a three-dimensional mixer, dry granulation was carried out, to obtain the moisture-proof acetyl L-carnitine.

### Example 3 Preparation of Moisture-Proof Acetyl L-Carnitine

A moisture-proof acetyl L-carnitine comprises the following components in percentage by weight:

| | |
|---|---|
| acetyl L-carnitine | 70% |
| mannitol | 15% |
| microcrystalline cellulose | 10% |
| magnesium stearate | 5%. |

Acetyl L-carnitine and mannitol were sieved with a 30-mesh sieve, acetyl L-carnitine, mannitol, microcrystalline cellulose and magnesium stearate of prescribed amounts were weighed and put into a three-dimensional mixer, dry granulation was carried out, to obtain the moisture-proof acetyl L-carnitine.

### Example 4 Preparation of Moisture-Proof Acetyl L-Carnitine

A moisture-proof acetyl L-carnitine comprises the following components in percentage by weight:

| | |
|---|---|
| acetyl L-carnitine | 80% |
| mannitol | 10% |
| microcrystalline cellulose | 5% |
| magnesium stearate | 5%. |

Acetyl L-carnitine and mannitol were sieved with a 30-mesh sieve, acetyl L-carnitine, mannitol, microcrystalline cellulose and magnesium stearate of prescribed amounts were weighed and put into a three-dimensional mixer, dry granulation was carried out, to obtain the moisture-proof acetyl L-carnitine.

### Example 5 Preparation of Moisture-Proof Acetyl L-Carnitine Hydrochloride

A moisture-proof acetyl L-carnitine hydrochloride comprises the following components in percentage by weight:

| | |
|---|---|
| acetyl L-carnitine hydrochloride | 80% |
| mannitol | 10% |
| microcrystalline cellulose | 5% |
| magnesium stearate | 5%. |

Acetyl L-carnitine hydrochloride and mannitol were sieved with a 30-mesh sieve, acetyl L-carnitine hydrochloride, mannitol, microcrystalline cellulose and magnesium stearate of prescribed amounts were weighed and put into a three-dimensional mixer, dry granulation was carried out, to obtain the moisture-proof acetyl L-carnitine hydrochloride.

### Example 6 Preparation of Moisture-Proof Acetyl L-Carnitine and Acetyl L-Carnitine Hydrochloride Mixture

A moisture-proof acetyl L-carnitine and acetyl L-carnitine hydrochloride mixture (i.e., a moisture-proof acetyl L-carnitine or hydrochloride thereof) comprises the following components in percentage by weight:

| | |
|---|---|
| acetyl L-carnitine | 40% |
| acetyl L-carnitine hydrochloride | 40% |
| mannitol | 10% |
| microcrystalline cellulose | 5% |
| magnesium stearate | 5%. |

Acetyl L-carnitine, acetyl L-carnitine hydrochloride and mannitol were sieved with a 30-mesh sieve, acetyl L-carnitine, acetyl L-carnitine hydrochloride, mannitol, microcrystalline cellulose and magnesium stearate of prescribed amounts were weighed and put into a three-dimensional mixer, dry granulation was carried out, to obtain the moisture-proof acetyl L-carnitine and acetyl L-carnitine hydrochloride mixture.

### Example 7 Investigation on Moisture-Proof Performance of Moisture-Proof Acetyl L-Carnitine or Hydrochloride Thereof Samples

A proper amount of test sample was weighed and put in a dry glass bottle (with an outer diameter of 50 mm and a height of 100 mm) opening, placed (standing) in a box with a temperature of 40° C.±2° C. and a relative humidity of 75%±5%. At regular intervals (at 0.5 h, 1 h, 1.5 h, 2 h, 3 h, 5 h and 7 h respectively), the glass bottle was taken out and the passing rate through a 24-mesh sieve and moisture content of the sample were detected. The passing rate was calculated according to the formula:

$$passing rate = 100 \% \times \frac{weight of material passing through sieve}{total weight of material} .$$

The test results of moisture-proof acetyl L-carnitine or hydrochloride thereof samples passing through a 24-mesh sieve after standing are shown in Table 1.

TABLE 1

| Sample | 0 h | 0.5 h | 1 h | 1.5 h | 2 h | 3 h | 5 h | 7 h |
|---|---|---|---|---|---|---|---|---|
| Raw material | 100.0% | 32.4% | 10.3% | 1.9% | 1.3% | 0.7% | 0.3% | 0.0% |
| Example 1 | 100.0% | 98.1% | 96.4% | 95.9% | 94.8% | 94.4% | 93.9% | 90.4% |
| Example 2 | 100.0% | 97.1% | 95.9% | 94.7% | 94.0% | 93.6% | 92.1% | 85.4% |
| Example 3 | 100.0% | 97.9% | 94.5% | 92.5% | 90.4% | 90.1% | 84.6% | 80.1% |
| Example 4 | 100.0% | 89.7% | 87.1% | 83.2% | 81.7% | 81.0% | 75.4% | 70.3% |
| Example 5 | 100.0% | 93.0% | 91.4% | 90.1% | 88.8% | 87.9% | 87.0% | 85.1% |
| Example 6 | 100.0% | 91.5% | 89.8% | 87.2% | 85.9% | 84.7% | 84.0% | 83.3% |

The test results of moisture content of moisture-proof acetyl L-carnitine or hydrochloride thereof samples after standing are shown in Table 2.

TABLE 2

| Sample | 0 h | 0.5 h | 1 h | 1.5 h | 2 h | 3 h | 5 h | 7 h |
|---|---|---|---|---|---|---|---|---|
| Raw material | 0.5% | 15.3% | 30.4% | 36.8% | 42.4% | 43.4% | 45.8% | 46.3% |
| Example 1 | 0.5% | 0.6% | 1.4% | 2.0% | 2.4% | 2.8% | 3.0% | 3.2% |
| Example 2 | 0.5% | 0.8% | 2.0% | 2.4% | 3.3% | 4.5% | 4.7% | 5.2% |
| Example 3 | 0.5% | 0.7% | 1.6% | 2.8% | 3.4% | 4.6% | 4.8% | 5.6% |
| Example 4 | 0.5% | 0.7% | 1.9% | 2.7% | 3.6% | 4.5% | 4.7% | 5.8% |
| Example 5 | 0.4% | 0.6% | 1.7% | 2.5% | 3.5% | 4.4% | 4.5% | 5.1% |
| Example 6 | 0.5% | 0.7% | 1.9% | 2.8% | 3.5% | 4.5% | 4.8% | 5.3% |

As can be seen from Table 1 and Table 2, compared with raw material, the moisture-proof performance of moisture-proof acetyl L-carnitine or hydrochloride thereof samples of various examples of the present invention is obviously improved and excellent under the conditions of temperature of 40° C.±2° C. and relative humidity of 75%±5%, while having a high load of not less than 50%; the passing rate of these samples through a 24-mesh sieve is high and the increase of moisture content is small. After standing for 0.5 h at temperature of 40° C.±2° C. and relative humidity of 75%±5%, the passing rate through a 24-mesh sieve is not less than 85%; after standing for 3 h at temperature of 40° C.±2° C. and relative humidity of 75%±5%, the passing rate through a 24-mesh sieve is not less than 80%; after standing for 5 h at temperature of 40° C.±2° C. and relative humidity of 75%±5%, the passing rate through a 24-mesh sieve is not less than 75%; after standing for 7 h at temperature of 40° C.±2° C. and relative humidity of 75%±5%, the passing rate through a 24-mesh sieve is not less than 70%. After standing for 0.5 h at temperature of 40° C.±2° C. and relative humidity of 75%±5%, the increase of moisture content is not more than 1.0%; after standing for 1 h at temperature of 40° C.±2° C. and relative humidity of 75%±5%, the increase of moisture content is not more than 2.0%; after standing for 2 h at temperature of 40° C.±2° C. and relative humidity of 75%±5%, the increase of moisture content is not more than 4.0%; after standing for 5 h at temperature of 40° C.±2° C. and relative humidity of 75%±5%, the increase of moisture content is not more than 5.0%.

In addition, the auxiliary materials screened in the dry granulation preparation process are beneficial to tableting and granulation, and the material fluidity is good. When the moisture-proof acetyl L-carnitine or hydrochloride thereof of the invention comprises, by weight, not less than 10%, 12%, 14%, 15%, 16%, 18%, 20%, 22%, 24%, 25%, 26%, 28%, 30%, 32%, 34%, 35%, 36%, 38%, 40%, 42%, 44%, 45%, 46%, 48%, 50%, 52%, 55%, 58%, 60%, 62%, 65%, 68%, 70%, 72%, 75%, 78%, 80%, 85%, 90%, 95%, 97%, 99% of acetyl L-carnitine, hydrochloride thereof, or the mixture of both, or comprises, by weight, 10-99%, 15-98%, 20-97%, 25-96%, 30-95%, 35-94%, 40-93%, 45-92%, 50-90% of acetyl L-carnitine, hydrochloride thereof, or the mixture of both, it can achieve more obvious moisture-proof and deliquescence resistance and fluidity enhancement than raw material under high humidity conditions.

Under the condition of ≤25° C. and ≤70% RH, the repose angle of each sample (before standing) was measured by using the fixed funnel injection method according to the following steps: (1) Apparatus preparation: Vertically fix the funnel above a flat circular disk, and adjust the exact height so that the diameter of the resulting powder cone can be controlled. (2) Adding material: Block the funnel outlet with a stopper rod, pour a sufficient amount of sample into the funnel, then withdraw the stopper rod, allowing the powder to fall freely onto the center of the disk to form a symmetrical cone. (3) Powder accumulation treatment: Gently tap the funnel wall or lightly stir with a thin rod, without directly vibrating or impacting the disk. Once the material has completely ceased flowing and the cone has stabilized, the measurement is carried out. (4) Use a protractor to directly measure the angle between the cone slope and the horizontal plane, or measure the cone height (h) and base radius (r) and calculate the angle. The results are shown in Table 3.

TABLE 3

| Sample | Repose angle |
|---|---|
| Raw material | 53.1° |
| Example 1 | 41.2° |
| Example 2 | 43.4º |
| Example 3 | 44.5° |
| Example 4 | 45.0° |
| Example 5 | 42.9º |
| Example 6 | 43.8° |

As can be seen from Table 3, the repose angle of each of the moisture-proof acetyl L-carnitine or hydrochloride thereof samples of various examples of the present invention is not more than 45°, 48°, 50°, while the repose angle of the raw material is more than 53°. The products prepared in Examples of the invention have good moisture-proof property.

Although specific embodiments and examples of the present disclosure have been illustrated herein, it will be appreciated by those skilled in the art that any modifications and variations can be made without departing from the spirit of the present disclosure. The examples and illustrations above are not intended to limit the scope of the present disclosure. Any combination of embodiments of the present disclosure, along with any obvious their extension or analogs, are within the scope of the present disclosure. Further, it is intended that the present disclosure encompass any arrangement, which is calculated to achieve that same purpose, and all such variations and modifications fall within the scope of the appended claims.

What is claimed is:

1. A moisture-proof acetyl L-carnitine or hydrochloride thereof, wherein the moisture-proof acetyl L-carnitine or hydrochloride thereof comprises, by weight, not less than 10% of acetyl L-carnitine, hydrochloride thereof, or the mixture of both.

2. The moisture-proof acetyl L-carnitine or hydrochloride thereof of claim 1, wherein the moisture-proof acetyl L-carnitine or hydrochloride thereof comprises, by weight, not less than 15%, 20%, 25%, 30%, 35%, 40%, 45%, 50% of acetyl L-carnitine, hydrochloride thereof, or the mixture of both.

3. The moisture-proof acetyl L-carnitine or hydrochloride thereof of claim 1, wherein the moisture-proof acetyl L-carnitine or hydrochloride thereof comprises, by weight, 20-98% of acetyl L-carnitine, hydrochloride thereof, or the mixture of both; 1-60% of one or more auxiliary materials selected from microcrystalline cellulose, compressible starch, lactose or mannitol; 1-20% of one or more auxiliary materials selected from silicon dioxide, magnesium stearate, calcium hydrogen phosphate, calcium silicate, micro-powder silica gel.

4. The moisture-proof acetyl L-carnitine or hydrochloride thereof of claim 1, wherein the moisture-proof acetyl L-carnitine or hydrochloride thereof comprises, by weight, 40-90% of acetyl L-carnitine, hydrochloride thereof, or the mixture of both; 2-50% of one or more auxiliary materials selected from microcrystalline cellulose, mannitol; 2-10% of one or more auxiliary materials selected from silicon dioxide, magnesium stearate.

5. The moisture-proof acetyl L-carnitine or hydrochloride thereof of claim 1, wherein the moisture-proof acetyl L-carnitine or hydrochloride thereof comprises, by weight, 50-80% of acetyl L-carnitine, hydrochloride thereof, or the mixture of both; 5-45% of one or more auxiliary materials selected from microcrystalline cellulose, mannitol; 2-5% of magnesium stearate.

6. The moisture-proof acetyl L-carnitine or hydrochloride thereof of claim 1, wherein the moisture-proof acetyl L-carnitine or hydrochloride thereof is in granular or powdery form.

7. The moisture-proof acetyl L-carnitine or hydrochloride thereof of claim 6, wherein the particle size of the moisture-proof acetyl L-carnitine or hydrochloride thereof is 10-100 meshes.

8. The moisture-proof acetyl L-carnitine or hydrochloride thereof of claim 1, wherein the moisture-proof acetyl L-carnitine or hydrochloride thereof is formulated into nutritional supplement, food, beverage or animal feed.

9. The moisture-proof acetyl L-carnitine or hydrochloride thereof of claim 1, wherein the moisture-proof acetyl L-carnitine or hydrochloride thereof is prepared into solid preparation or liquid preparation.

10. The moisture-proof acetyl L-carnitine or hydrochloride thereof of claim 1, wherein the passing rate of the moisture-proof acetyl L-carnitine or hydrochloride thereof through a 24-mesh sieve is not less than 70% after standing for a period of time.

11. The moisture-proof acetyl L-carnitine or hydrochloride thereof of claim 1, wherein the passing rate of the moisture-proof acetyl L-carnitine or hydrochloride thereof through a 24-mesh sieve is not less than 85% after standing for 0.5 h.

12. The moisture-proof acetyl L-carnitine or hydrochloride thereof of claim 1, wherein the passing rate of the moisture-proof acetyl L-carnitine or hydrochloride thereof through a 24-mesh sieve is not less than 80% after standing for 3 h.

13. The moisture-proof acetyl L-carnitine or hydrochloride thereof of claim 1, wherein the passing rate of the moisture-proof acetyl L-carnitine or hydrochloride thereof through a 24-mesh sieve is not less than 75% after standing for 5 h.

14. The moisture-proof acetyl L-carnitine or hydrochloride thereof of claim 1, wherein the passing rate of the moisture-proof acetyl L-carnitine or hydrochloride thereof through a 24-mesh sieve is not less than 70% after standing for 7 h.

15. The moisture-proof acetyl L-carnitine or hydrochloride thereof of claim 1, wherein the increase of moisture content of the moisture-proof acetyl L-carnitine or hydrochloride thereof is not more than 5.0% after standing for a period of time.

16. The moisture-proof acetyl L-carnitine or hydrochloride thereof of claim 1, wherein the increase of moisture content of the moisture-proof acetyl L-carnitine or hydrochloride thereof is not more than 1.0% after standing for 0.5 h.

17. The moisture-proof acetyl L-carnitine or hydrochloride thereof of claim 1, wherein the increase of moisture content of the moisture-proof acetyl L-carnitine or hydrochloride thereof is not more than 2.0% after standing for 1 h.

18. The moisture-proof acetyl L-carnitine or hydrochloride thereof of claim 1, wherein the increase of moisture content of the moisture-proof acetyl L-carnitine or hydrochloride thereof is not more than 4.0% after standing for 2 h.

19. The moisture-proof acetyl L-carnitine or hydrochloride thereof of claim 1, wherein the increase of moisture content of the moisture-proof acetyl L-carnitine or hydrochloride thereof is not more than 5.0% after standing for 5 h.

20. The moisture-proof acetyl L-carnitine or hydrochloride thereof of claim 1, wherein the repose angle of the moisture-proof acetyl L-carnitine or hydrochloride thereof is not more than 45°.
